(19) United States
(12) Patent Application Publication
(10) Pub. No.: US 2011/0263431 A1
(43) Pub. Date: Oct. 27, 2011

(54) PYRROLE DERIVATIVES FOR USE AS PLANT GROWTH REGULATORS

(75) Inventors: Camilla Corsi, Stein (CH); Sebastian Volker Wendeborn, Stein (CH); Carla Bobbio, Stein (CH); Jilali Kessabi, Stein (CH); Peter Schneiter, Stein (CH); Valeria Grasso, Stein (CH); Ulrich Johannes Haas, Stein (CH)

(73) Assignee: SYNGENTA CROP PROTECTION LLC, Greensboro, NC (US)

(21) Appl. No.: 13/140,787

(22) PCT Filed: Dec. 11, 2009
(86) PCT No.: PCT/EP2009/066961

§ 371 Date: Jun. 17, 2011

(30) Foreign Application Priority Data
Dec. 17, 2008 (GB) 0823003.9

(51) Int. Cl. A01N 43/54; C07D 403/06; A01P 21/00; C07D 405/14; A01N 43/40; C07D 401/06; C07D 409/14

(52) U.S. Cl. 504/242; 546/276.4; 544/333; 544/319; 504/252; 504/239

(57) ABSTRACT

The present invention relates to pyrrole compounds of formula (I) having plant growth regulating properties, to agricultural compositions comprising them, and to the use of said compounds for regulating plant growth.

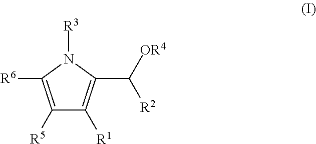

The present invention relates to pyrrole derivatives with plant growth regulating properties, to compositions which contain them, and to the use of said derivatives for regulating plant growth.

Plant growth regulators (PGRs) are generally any substances or mixtures of substances intended to accelerate or retard the rate of growth or maturation, or otherwise alter the development of plants or their produce. PGRs affect growth and differentiation of plants, a process which is commonly and hereinafter referred to as “plant health”. There exists a need for further substances having PGR activity.

International patent application WO2008/155081 refers to a series of substituted pyrrole-containing compounds having fungicidal properties.

It has surprisingly been found that the substituted pyrrole-containing compounds of the present invention exhibit plant growth regulating properties and are therefore suitable for use in agriculture for the improvement and control of plant health.

Accordingly, in a first aspect, the present invention provides for the use of a compound of the formula (I)

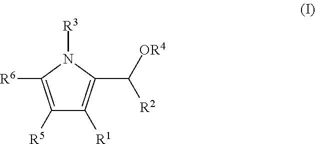

wherein
R¹ and R³ are independently hydrogen or optionally substituted alkyl, alkenyl, alkynyl, heterocyclyl, trialkylsilyl, arylalkyl, aryloxyalkyl, arylthioalkyl, aryl or heteroaryl;
R² is optionally substituted alkyl, alkenyl, alkynyl, heterocyclyl, arylalkyl, aryl or heteroaryl;
R⁴ is H or acyl;
R⁵ and R⁶ are, independently, hydrogen, cyano, halogen or optionally substituted alkyl, alkenyl, alkynyl, heterocyclyl, alkoxy, alkoxycarbonyl, alkylthio, trialkylsilyl, arylalkyl, aryloxyalkyl, arylthioalkyl, aryl or heteroaryl;
or a salt or N-oxide thereof; as a plant growth regulator.

Plant growth regulators can, for example, reduce plant height, stimulate seed germination, induce flowering, darken leaf coloring, change the rate of plant growth and modify the timing and efficiency of fruiting. In addition, PGRs may exhibit pronounced growth-regulating properties which can result in an increase in the yield of cultivated plants or harvested crops.

PGRs may also have a growth inhibiting action which is dependent on concentration. The growth of both monocots and dicots may be inhibited. Inhibition of the vegetative growth of many cultivated plants permits more plants to be sown in a crop area, so that a higher yield may be obtained per unit of area. Inhibition of the vegetative growth of monocot plants, e.g. cultivated plants such as cereals, is sometimes desirable and advantageous. Such a growth inhibition is of economic interest.

The use of PGRs for inhibiting the growth in height of cereals is also important, as shortening the stalks diminishes or completely eliminates the danger of lodging before harvesting. Additionally, PGRs are able to bring about a strengthening of the stalks in crops of cereals and this too counteracts lodging.

Furthermore, the present invention also provides compositions comprising the pyrrole derivatives of the present invention that improve plants, a process which is commonly and hereinafter referred to as “plant health”.

For example, advantageous properties that may be mentioned are improved crop characteristics including: emergence, crop yield, protein content, increased vigour, faster/delayed maturation, increased speed of seed emergence, improved nutrient utilization efficiency, improved nitrogen utilization efficiency, improved water use efficiency, improved oil content and for quality, improved digestibility, faster/more even ripening, improved flavor, improved starch content, more developed root system (improved root growth), improved stress tolerance (e.g. against drought, heat, salt, light, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Advantageous properties obtained, especially from treated seeds, include, for example, improved germination and field establishment, better vigor and more homogeneous field establishment.

Advantageous properties obtained, especially from foliar and/or in-furrow application include, for example, improved plant growth and plant development, better growth, more tillers, greener leafes, largers leaves, more biomass, better roots, improved stress tolerance of the plants, more grain yield, more biomass harvested, improved quality of the harvest (content of fatty acids, metabolites, oil etc), more marketable products (e.g. improved size), improved process (e.g. longer shelf-life, better extraction of compounds), improved quality of seeds (for being seeded in the following seasons for seed production); or any other advantages familiar to a person skilled in the art.

It is therefore an object of the present invention to provide compositions and methods suitable for addressing the opportunities outlined above.

The present invention provides plant-protecting active ingredients that are the pyrrole compounds of formula (I) according to the invention, in particular the individual pyrrole compounds described in the description as being preferred, and mixtures with increased efficacy and to a method of improving the health of plants by applying said compounds and mixtures to the plants or the locus thereof.

The action of the compounds of formula (I) is separate to any fungicidal action. The pyrrole compounds of formula (I) according to the invention, in particular the individual pyrrole compounds described in the above description as being preferred compounds exhibit plant health properties.

The present invention also concerns compositions comprising or consisting essentially of an active compound as described herein in combination with a suitable carrier (e.g., an agricultural carrier).

The foregoing and other objects and aspects of the present invention are explained in greater detail below.

“Alkyl” as used herein refers to a saturated hydrocarbon radical which may be straight-chain or branched-chain or cyclic (cycloalkyl) and contains from 1 to 24 carbon atoms. This definition applies both when the term is used alone and when it is used as part of a compound term, such as haloalkyl and similar terms. Preferred straight chain and branched alkyl groups may contain 1 to 8 carbon atoms, more preferably 1 to 4 carbons, even more preferably, 1 to 4 carbon atoms. Representative alkyl groups include, for example, methyl, ethyl, isopropyl, n-propyl, n-butyl, t-butyl, t-amyl, and 2,5-dimethylhexyl. Preferred cycloalkyl groups may contain 3 to 12 carbon atoms, more preferably 4 to 10 carbons, even more preferably, 5 to 8 carbon atoms and most preferably 5 or 6 carbon atoms. Preferred cycloalkyl groups include, for example, cyclobutyl, cyclopropyl, cyclopentyl and cyclohexyl.

“Alkenyl” as used herein, refers to a straight or branched chain hydrocarbon containing from 2 to 24 carbons, more preferably 2 to 8 carbons, yet more preferably, 2 to 6 carbon atoms, even more preferably 2 to 4 carbon atoms, and containing at least one carbon-carbon double bond. Representative alkenyl groups include, for example, ethenyl, 2-propenyl, 2-methyl-2-propenyl, 3-butenyl, 4-pentenyl, 5-hexenyl, 2-heptenyl, 2-methyl-1-heptenyl and 3-decenyl.

“Alkynyl” as used herein, refers to a straight or branched chain hydrocarbon group containing from 2 to 24 carbons, more preferably 2 to 8 carbons, yet more preferably, 2 to 6 carbon atoms, even more preferably 2 to 4 carbon atoms, and containing at least one carbon-carbon triple bond. Representative alkynyl groups include, for example, acetylenyl, 1-propynyl, 2-propynyl, 3-butynyl, 2-pentynyl and 1-butynyl.

Representative alkoxy groups include, for example, methoxy, ethoxy and t-butoxy.

Representative alkylthio groups include, for example, methylthio, ethylthio, t-butylthio and hexylthio.

“Aryl” refers to an aromatic substituent which may be a single ring or multiple rings which are fused together, linked covalently or linked to a common group such as an ethylene or methylene moiety. The aromatic rings may each contain heteroatoms and hence aryl encompasses heteroaryl as used herein. Aryl moieties may be optionally substituted with 1 to 4 substituents independently selected from halogen, nitro, alkylcarboxyl, alkoxy and phenoxy. Representative examples of aryl include phenyl azulenyl, indanyl, indenyl, naphthyl, tetrahydronaphthyl, biphenyl, diphenylmethyl, 2,2-diphenyl-1-ethyl, thienyl, pyridyl and quinoxalyl. Most preferably, aryl is phenyl.

“Heteroaryl” means a cyclic, aromatic hydrocarbon containing 3 to 10 ring-atoms including 1 to 4 heteroatoms independently selected from nitrogen, oxygen and sulfur. Preferred heteroaryl groups are five and six membered rings and contain from one to three heteroatoms independently selected from nitrogen, oxygen and sulphur. Heteroaryl moieties may be optionally substituted with 1 to 4 substituents independently selected from halogen, nitro, alkylcarboxyl, alkoxy and phenoxy. Examples of heteroaryl groups include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyrimidinyl, pyrazinyl, pyranyl, pyridazinyl, tetrazolyl, triazinyl.

In addition, the term heteroaryl includes fused heteroaryl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, benzothiazolyl, phthalimido, benzofuranyl, benzodiazepinyl, indolyl, isoindolyl, isobenzofuranyl, chromenyl, xanthenyl, indolizinyl, indazolyl, purinyl, quinolizinyl, isoquinolyl, phthalazinyl, naphthyridinyl and benzo[b]thienyl.

“Heterocyclyl”, as used herein refers to a saturated or partially unsaturated cyclic hydrocarbon containing from 3 to 10 ring-atoms up to 4 of which may be hetero-atoms such as nitrogen, oxygen and sulfur. Examples of heterocyclyl groups are oxiranyl, azetidinyl, tetrahydrofuranyl, thiolanyl, pyrrolidinyl, pyrrolinyl, imidazolidinyl, imidazolinyl, sulfolanyl, dioxolanyl, dihydropyranyl, tetrahydropyranyl, piperidinyl, pyrazolinyl, pyrazolidinyl, dioxanyl, morpholinyl, dithianyl, thiomorpholinyl, piperazinyl, azepinyl, oxazepinyl, thiazepinyl, thiazolinyl and diazapanyl.

Acyl includes any readily hydrolysable acyl groups, and comprises, for example, C(O)R⁷, C(O)OR⁷, C(O)NHR⁷ and C(O)NR⁷R⁸, wherein R⁷ and R⁸ are each independently selected from alkyl, alkenyl, akynyl, heterocyclyl, aryl and heteroaryl. Acyl groups may be optionally substituted with one or more, for example 1, 2, 3 or 4, halo or OR⁷ groups. Preferred acyl groups are acetyl, benzoyl and phenylacetyl.

“Halo” or “halogen” means fluoro, chloro, bromo and iodo and is preferably fluoro or chloro.

“Haloalkyl” includes monohaloalkyl, polyhaloalkyl and perhaloalkyl, for example, chloromethyl, 2-bromoethyl, 2-fluoroethyl, 2,2,2-trifluoroethyl, chlorodifluoromethyl, trichloromethyl, trifluoromethyl, pentafluoroethyl and 2-chloro-3-fluoropentyl.

“Organic base” as used herein includes, for example, triethylamine, triisobutylamine, triiooctylamine, triisodecylamine, diethanolamine, triethanolamine, pyridine, morpholine, and mixtures thereof. A preferred category of organic base is organic amines.

“Inorganic base” as used herein includes, for example, sodium carbonate, sodium bicarbonate, potassium carbonate, and mixtures thereof.

“Optionally substituted” means substituted by one or more substituents, in particular, one, two, three or four substituents, independently selected from halogen, hydroxyl, cyano, nitro, alkyl, haloalkyl, alkenyl, haloalkenyl, alkynyl, haloalkynyl, heterocyclyl, aryl, heteroaryl, alkoxy, haloalkoxy, alkylthio, haloalkylthio, acyl, alkoxycarbonyl and trialkylsilyl. In the case where groups may be selected from a number of alternative groups, the selected groups may be the same or different.

“Agriculturally acceptable salt” means a salt the cation of which is known and accepted in the art for the formation of salts for agricultural or horticultural use. Preferably the salts are water-soluble.

The compounds of formula (I) may exist in different geometric or optical isomeric forms or in different tautomeric forms. One or more centres of chirality may be present, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers of mixtures of isomers. Centres of tautomerisation may be present. This invention covers all such isomers and tautomers and mixtures thereof in all proportions as well as isotopic forms such as deuterated compounds.

Suitable salts of the compounds of formula (I) include acid addition salts such as those with an inorganic acid such as hydrochloric, hydrobromic, sulphuric, nitric or phosphoric acid, or an organic carboxylic acid such as oxalic, tartaric, lactic, butyric, toluic, hexanoic or phthalic acid, or a sulphonic acid such as methane, benzene or toluene sulphonic acid. Other examples of organic carboxylic acids include haloacids such as trifluoroacetic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

In another aspect, the present invention provides a method of regulating plant growth of crops of useful plants, which comprises applying to said plants, to one or more parts of said plants, or to the locus thereof or plant propagation material, a compound of formula (I) as defined herein.

The preferred embodiments of the invention as defined below apply equally to each aspect and preferred aspects thereof of the invention as defined herein.

In a preferred embodiment, R¹ is selected from hydrogen; alkyl optionally substituted with phenyl or halophenyl; aryl optionally substituted with halogen, alkyl, haloalkyl, alkoxy, alkylthio, haloalkoxy, cyano or nitro; and heteroaryl optionally substituted with halogen, alkyl, haloalkyl, alkoxy, alkylthio, haloalkoxy, cyano or nitro; or trialkylsilyl.

Preferably, R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

More preferably, R¹ is selected from hydrogen, methyl, ethyl, n-propyl, isopropyl, isoamyl, cyclohexyl, benzyl; phenyl optionally substituted with 1 to 3 groups independently selected from F, Cl, Br, methyl, ethyl, methoxy, ethoxy, trifluoromethyl and trifluoromethoxy; and pyridyl, furyl, thienyl or pyrimidinyl optionally substituted with 1 to 3 groups independently selected from F, Cl, Br, methyl, ethyl, methoxy, ethoxy, trifluoromethyl and trifluoromethoxy.

Yet more preferably, R¹ is selected from n-propyl, isopropyl, cyclohexyl, benzyl; phenyl optionally substituted with 1 or 2 groups independently selected from bromo, chloro, fluoro, methyl, methoxy, trifluoromethyl and trifluoromethoxy; and 2- or 3-pyridyl, 2- or 3-furyl, and 2- or 3-thienyl, each optionally substituted with 1 or 2 chloro.

Most preferably, R¹ is selected from 2-chlorophenyl, 3-chlorophenyl, 4-chlorophenyl, 4-bromophenyl, 2-fluorophenyl, 4-fluorophenyl, 2,4-dichlorophenyl, 2,4-difluorophenyl, 2-fluoro-4-chlorophenyl, 2-chloro-4-fluorophenyl, 2-methylphenyl, 4-methylphenyl, 2,4-dimethylphenyl, 2-methoxyphenyl, 4-methoxyphenyl, 3-trifluoromethylphenyl, 4-trifluoromethylphenyl, 2-chloro-4-methoxyphenyl, 4-methoxytrifluomethylphenyl, 2-methyl-4-chlorophenyl, 2-chloro-3-pyridyl, 2-methoxy-3-pyridyl, 2-thienyl, 3-thienyl and 5-chloro-2-thienyl.

In a preferred embodiment, R² is heteroaryl optionally substituted with halogen, alkyl, alkenyl, alkynyl, haloalkyl, alkoxy, alkylthio, haloalkoxy, cyano or nitro.

Preferably, R² is pyridyl or pyrimidinyl, each optionally substituted with halogen, C₁-C₆-alkyl, C₁-C₆-haloalkyl, C₁-C₆-alkoxy or C₁-C₆-haloalkoxy.

More preferably, R² is selected from 2-, 3- or 4-pyridyl and 5-pyrimidinyl, each optionally substituted with halogen, C₁-C₆-haloalkyl or C₁-C₆-alkoxy.

Yet more preferably, R² is selected from 2-pyridyl, 3-pyridyl, and 5-pyrimidinyl, each optionally substituted with methyl, chloro, fluoro or methoxy.

Most preferably, R² is 3-pyridyl.

In another preferred embodiment, R³ is selected from hydrogen; alkyl optionally substituted with phenyl or halophenyl: aryl optionally substituted with halogen, alkyl, haloalkyl, alkoxy, alkylthio, haloalkoxy, cyano or nitro; heteroaryl optionally substituted with halogen, alkyl, haloalkyl, alkoxy, alkylthio, haloalkoxy, cyano or nitro; and trialkylsilyl.

Preferably, R³ is selected from hydrogen; C₁-C₆-alkyl optionally substituted with phenyl: phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

More preferably, R³ is selected from hydrogen, methyl, ethyl, n-propyl, isopropyl, isoamyl cyclohexyl, benzyl; phenyl optionally substituted with 1 to 3 groups independently selected from bromo, chloro, fluoro, methyl, ethyl, methoxy, ethoxy, trifluoromethyl and trifluoromethoxy; and pyridyl, furyl, thienyl and pyrimidinyl, each optionally substituted with 1 to 3 groups independently selected from bromo, chloro, fluoro, methyl, ethyl, methoxy, ethoxy, trifluoromethyl and trifluoromethoxy.

Yet more preferably, R³ is selected from ethyl, isopropyl, isoamyl, cyclohexyl; phenyl optionally substituted with 1 or 2 groups independently selected from chloro, fluoro and trifluoromethyl; and 2- or 3-furyl, and 2- or 3-thienyl, each optionally substituted with 1 or 2 chloro.

Most preferably, R³ is 3-chlorophenyl.

In a preferred embodiment, R⁴ is selected from H, acetyl, C(O)Et and C(O)ⁱPr. Most preferably, R⁴ is H.

In a preferred embodiment, R⁵ is selected from hydrogen, halogen, C₁-C₆-alkyl, C₁-C₆-alkoxy, cyano; and phenyl optionally substituted with halogen, C₁-C₆-alkyl, C₁-C₆-haloalkyl, C₁-C₆-alkoxy or C₁-C₆-haloalkoxy.

More preferably, R⁵ is selected from hydrogen, halogen, C₁-C₄-alkyl, C₁-C₄-alkoxy, cyano; and phenyl optionally substituted with halogen.

Yet more preferably, R⁵ is selected from hydrogen, chloro, fluoro, methyl, ethyl, butyl, methoxy, cyano; and phenyl optionally substituted with chloro or fluoro.

Most preferably, R⁵ is hydrogen.

In a preferred embodiment, R⁶ is selected from hydrogen, halogen, C₁-C₆-alkyl, C₁-C₆-alkoxy, cyano; and phenyl optionally substituted with halogen, C₁-C₆-alkyl, C₁-C₆-haloalkyl, C₁-C₆-alkoxy or C₁-C₆-haloalkoxy.

More preferably, R⁶ is selected from hydrogen, halogen, C₁-C₄-alkyl, C₁-C₄-alkoxy, cyano; and phenyl optionally substituted with halogen.

Yet more preferably, R⁶ is selected from hydrogen, chloro, fluoro, methyl, ethyl, butyl, methoxy, cyano; and phenyl optionally substituted with chloro or fluoro.

Most preferably, R⁶ is hydrogen.

In one preferred aspect of the present invention:

R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy;
R² is heteroaryl optionally substituted with halogen, alkyl, alkenyl, alkynyl, haloalkyl, alkoxy, alkylthio, haloalkoxy, cyano or nitro;
R³ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy;

### R⁴ is H;

R⁵ is selected from hydrogen, halogen, C₁-C₄-alkyl, C₁-C₄-alkoxy and cyano; and phenyl optionally substituted with halogen; and
R⁶ is selected from hydrogen, halogen, C₁-C₄-alkyl, C₁-C₄-alkoxy and cyano; and phenyl optionally substituted with halogen.

In a more preferred aspect of the present invention:

R¹ is selected from n-propyl, isopropyl, cyclohexyl, benzyl; phenyl optionally substituted with 1 or 2 groups independently selected from bromo, chloro, fluoro, methyl, methoxy, trifluoromethyl and trifluoromethoxy; and 2- or 3-pyridyl, 2- or 3-furyl, and 2- or 3-thienyl, each optionally substituted with 1 or 2 chloro;
R² is selected from 2-pyridyl, 3-pyridyl, and 5-pyrimidinyl, each optionally substituted with methyl, chloro, fluoro or methoxy;
R³ is selected from ethyl, isopropyl, isoamyl cyclohexyl; phenyl optionally substituted with 1 or 2 groups independently selected from chloro, fluoro and trifluoromethyl; and 2- or 3-furyl, and 2- or 3-thienyl, each optionally substituted with 1 or 2 chloro; and

### R⁴, R⁶ and R⁶ are H.

In a further aspect, the present invention provides a method of regulating plant growth of crops of useful plants, which comprises applying to said plants, to one or more parts of said plants, or to the locus thereof or plant propagation material, a compound of formula (I) as defined herein.

In a further aspect, the present invention provides a method of regulating plant growth of crops of useful plants, which comprises one or more applications of one of more compounds of formula (I) alone or in conjunction with one or more customary plant protection formulating auxiliaries.

In a further aspect, the present invention provides a method of regulating plant growth of crops of useful plants, which comprises applying to said plants, to one or more parts of said plants, or to the locus thereof or plant propagation material, a compound of formula (I) as defined herein, wherein two or more applications are carried out in sequence, and wherein the two or more applications have the same or different concentration or combinations of compounds as defined herein or both.

In a preferred embodiment of the invention, the crops of useful plants are selected from cereals, rice, beets, leguminous plants, oil plants, cucumber plants, fibre plants, vegetables, plantation crops, ornamentals, vines, bushberries, caneberries, cranberries, peppermint, rhubarb, spearmint, sugar cane and turf grasses.

In a preferred embodiment of the invention, the plant growth regulating effect is an inhibition or a retardation of the plant growth.

In an especially preferred embodiment, the present invention provides an agricultural composition comprising one or more compounds of formula (I) as defined herein and one or more customary plant protection auxiliaries.

In a further aspect, the present invention is directed to the (R)-enantiomers of the compounds of formula (I), designated (R)-(I), wherein R¹, R², R³, R⁴, R⁵ and R⁶ are as defined herein; and salts thereof.

Preferred compounds of formula (R)-(I) include the (R)-enantiomers of compounds 1 to 61 of Table 1 herein, 62 to 85 and 87 of Table 2 herein and 86 of Table 3 herein.

The present invention provides the compound of formula (R)-(I) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In another aspect, the present invention relates to an agricultural composition comprising a compound of formula (R)-(I), wherein R¹, R², R³, R⁴, R⁵ and R⁶ are as defined herein; or an agrochemically acceptable salt thereof, and an agrochemically acceptable diluent or carrier.

In a further aspect, the present invention is directed to the (S)-enantiomers of the compounds of formula (I), designated (S)-(I), wherein R¹, R², R³, R⁴, R⁵ and R⁶ are as defined herein; and salts thereof.

Preferred compounds of formula (S)-(I) include the (S)-enantiomers of compounds 1 to 61 of Table 1 herein, 62 to 85 and 87 of Table 2 herein and 86 of Table 3 herein.

The present invention provides the compound of formula (S)-(I) as a single enantiomer having an enantiomeric excess (e.e.) of at least 40%, for example, at least 50%, 60%, 70% or 80%, preferably at least 90%, more preferably at least 95%, yet more preferably at least 98% and most preferably at least 99%.

In another aspect, the present invention relates to an agricultural composition comprising a compound of formula (S)-(I), wherein R¹, R², R³, R⁴, R⁵ and R⁶ are as defined herein; or an agrochemically acceptable salt thereof, and an agrochemically acceptable diluent or carrier.

“Plant propagation material” means generative parts of a plant including seeds of all kinds (fruit, tubers, bulbs, grains etc), roots, rhizomes, cuttings, cut shoots and the like. Plant propagation material may also include plants and young plants which are to be transplanted after germination or after emergence from the soil.

“Locus” means the fields on which the plants to be treated are growing, or where the seeds of cultivated plants are sown, or the place where the seed will be placed into the soil.

The “crops of useful plants” to be protected typically comprise, for example, the following species of plants: cereals (wheat, barley, rye, oats, maize (including field corn, pop corn and sweet corn), rice, sorghum and related crops); beet (sugar beet and fodder beet); leguminous plants (beans, lentils, peas, soybeans); oil plants (rape, mustard, sunflowers); cucumber plants (marrows, cucumbers, melons); fibre plants (cotton, flax, hemp, jute); vegetables (spinach, lettuce, asparagus, cabbages, carrots, eggplants, onions, pepper, tomatoes, potatoes, paprika, okra); plantation crops (bananas, fruit trees, rubber trees, tree nurseries), ornamentals (flowers, shrubs, broad-leaved trees and evergreens, such as conifers); as well as other plants such as vines, bushberries (such as blueberries), caneberries, cranberries, peppermint, rhubarb, spearmint, sugar cane and turf grasses including, for example, cool-season turf grasses (for example, bluegrasses (Poa L.), such as Kentucky bluegrass (Poa pratensis L.), rough bluegrass (Poa trivialis L.), Canada bluegrass (Poa compressa L.) and annual bluegrass (Poa annua L.); bentgrasses (Agrostis L.), such as creeping bentgrass (Agrostis palustris Huds.), colonial bentgrass (Agrostis tenius Sibth.), velvet bentgrass (Agrostis canina L.) and redtop (Agrostis alba L.); fescues (Festuca L.), such as tall fescue (Festuca arundinacea Schreb.), meadow fescue (Festuca elatior L.) and fine fescues such as creeping red fescue (Festuca rubra L.), chewings fescue (Festuca rubra var. commutate Gaud.), sheep fescue (Festuca ovina L.) and hard fescue (Festuca longifolia); and ryegrasses (Lolium L.), such as perennial ryegrass (Lolium perenne L.) and annual (Italian) ryegrass (Lolium multiflorum Lam.)) and warm-season turf grasses (for example, Bermudagrasses (Cynodon L. C. Rich), including hybrid and common Bermudagrass; Zoysiagrasses (Zoysia Willd.), St. Augustinegrass (Stenotaphrum secundatum (Walt.) Kuntze); and centipedegrass (Eremochloa ophiuroides (Munro.) Hack.)).

The term “useful plants” also includes also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” also includes useful plants which have been so transformed by the use of recombinant DNA techniques that they are capable of synthesising one or more selectively acting toxins, such as are known, for example, from toxin-producing bacteria, especially those of the genus Bacillus.

The term “useful plants” also includes useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The agrochemical composition of the present invention will usually contain from 0.1 to 99% by weight, preferably from 0.1 to 95% by weight, of the compound of formula (I), 99.9 to 1% by weight, preferably 99.8 to 5% by weight, of a solid or liquid adjuvant, and from 0 to 25% by weight, preferably from 0.1 to 25% by weight, of a surfactant.

Suitably, the agrochemical compositions of the present invention are applied prior to disease development. Rates and frequency of use of the formulations are those conventionally used in the art and factors such as the developmental stage of the plant and on the location, timing and application method. Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds.

In practice, as indicated above, the agrochemical compositions comprising compound of formula (I) are applied as a formulation containing the various adjuvants and carriers known to or used in the industry. They may thus be formulated as granules, as wettable or soluble powders, as emulsifiable concentrates, as coatable pastes, as dusts, as flowables, as solutions, as suspensions or emulsions, or as controlled release forms such as microcapsules. These formulations are described in more detail below and may contain as little as about 0.5% to as much as about 95% or more by weight of the active ingredient. The optimum amount will depend on formulation, application equipment and nature of the plant to be treated.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from about 0.5% to about 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain about 5% to about 95% of the active ingredient plus a small amount of wetting, dispersing or emulsifying agent. Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from about 0.5% to about 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain about 5% to about 25% active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically about 1 to 50 microns in diameter. The enclosed liquid typically constitutes about 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art. Suitable examples of the different classes are found in the non-limiting list below.

Liquid carriers that can be employed include water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc. ethylene glycol, propylene glycol, glycerine, N-methyl-2-pyrrolidinone, and the like. Water is generally the carrier of choice for the dilution of concentrates.

Suitable solid carriers include talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour, lignin and the like.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants, sticking agents, and the like.

In addition, further, other biocidally active ingredients or compositions may be combined with the compound of formula (I) and used in the methods of the invention and applied simultaneously or sequentially with the compound of formula (I). When applied simultaneously, these further active ingredients may be formulated together with the compound of formula (I) or mixed in, for example, the spray tank. These further biocidally active ingredients may be fungicides, herbicides, insecticides, bactericides, acaricides, nematicides and/or plant growth regulators.

Accordingly, the present invention provides for the use of a composition in the methods of the present invention, said composition comprising (i) a compound of formula (I) as defined herein, or compounds 1 to 61 of Table 1 or 62 to 86 of Table 2 and (i) a fungicide, (ii) a herbicide, (iii) an insecticide, (iv) a bactericide, (v) an acaricide, (vi) a nematicide and/or (vii) a plant growth regulator.

In a further aspect, the present invention provides a composition comprising a compound of formula (I), which is the (R)-enantiomer of formula (R)-(I) and (i) a fungicide, (ii) a herbicide, (iii) an insecticide, (iv) a bactericide, (v) an acaricide, (vi) a nematicide and/or (vii) a plant growth regulator.

Additionally, the present invention provides for the use of a composition in the methods of the present invention, said composition comprising a compound of formula (I), which is the (R)-enantiomer of formula (R)-(I), and (i) a fungicide, (ii) a herbicide, (iii) an insecticide, (iv) a bactericide, (v) an acaricide, (vi) a nematicide and/or (vii) a plant growth regulator.

In a further aspect, the present invention provides a composition comprising a compound of formula (I), which is the (S)-enantiomer of formula (S)-(I) and (i) a fungicide, (ii) a herbicide, (iii) an insecticide, (iv) a bactericide, (v) an acaricide, (vi) a nematicide and/or (vii) a plant growth regulator.

Additionally, the present invention provides for the use of a composition in the methods of the present invention, said composition comprising a compound of formula (I), which is the (S)-enantiomer of formula (S)-(I), and (i) a fungicide, (ii) a herbicide, (iii) an insecticide, (iv) a bactericide, (v) an acaricide, (vi) a nematicide and/or (vii) a plant growth regulator.

In addition, the compounds of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-5-methyl.

In particular, composition encompassed by the present invention include, for example, compositions comprising a compound of formula (I) and acibenzolar (CGA245704), a compound of formula (I) and ancymidol, a compound of formula (I) and alanycarb, a compound of formula (I) and aldimorph, a compound of formula (I) and amisulbrom, a compound of formula (I) and anilazine, a compound of formula (I) and azaconazole, a compound of formula (I) and azoxystrobin, a compound of formula (I) and benalaxyl, a compound of formula (I) and benthiavalicarb, a compound of formula (I) and benomyl, a compound of formula (I) and biloxazol, a compound of formula (I) and bitertanol, a compound of formula (I) and bixafen, a compound of formula (I) and blasticidin S, a compound of formula (I) and boscalid, a compound of formula (I) and bromuconazole, a compound of formula (I) and bupirimate, a compound of formula (I) and captafol, a compound of formula (I) and captan, a compound of formula (I) and carbendazim, a compound of formula (I) and carbendazim, a compound of formula (I) and chlorhydrate, a compound of formula (I) and carboxin, a compound of formula (I) and carpropamid, a compound of formula (I) and carvone, a compound of formula (I) and CGA41396, a compound of formula (I) and CGA41397, a compound of formula (I) and chinomethionate, a compound of formula (I) and chloroneb, a compound of formula (I) and chlorothalonil, a compound of formula (I) and chlorozolinate, a compound of formula (I) and clozylacon, a compound of formula (I) and copper containing compounds such as copper oxychloride, copper oxyquinolate, copper sulphate, copper tallate and Bordeaux mixture, a compound of formula (I) and cyflufenamid, a compound of formula (I) and cymoxanil, a compound of formula (I) and cyproconazole, a compound of formula (I) and cyprodinil, a compound of formula (I) and debacarb, a compound of formula (I) and di-2-pyridyl disulphide 1,1′-dioxide, a compound of formula (I) and dichlofluanid, a compound of formula (I) and diclomezine, a compound of formula (I) and dichlozoline, a compound of formula (I) and dichlone, a compound of formula (I) and dicloran, a compound of formula (I) and diclocymet, a compound of formula (I) and diethofencarb, a compound of formula (I) and difenoconazole, a compound of formula (I) and difenzoquat, a compound of formula (I) and diflumetorim, a compound of formula (I) and O,O-di-iso-propyl-5-benzyl thiophosphate, a compound of formula (I) and dimefluazole, a compound of formula (I) and dimetconazole, a compound of formula (I) and dimethomorph, a compound of formula (I) and dimethirimol, a compound of formula (I) and dimoxystrobin, a compound of formula (I) and diniconazole, a compound of formula (I) and dinocap, a compound of formula (I) and dithianon, a compound of formula (I) and dodecyl dimethyl ammonium chloride, a compound of formula (I) and dodemorph, a compound of formula (I) and dodine, a compound of formula (I) and doguadine, a compound of formula (I) and edifenphos, a compound of formula (I) and enestrobin, a compound of formula (I) and epoxiconazole, a compound of formula (I) and ethaboxam, a compound of formula (I) and ethirimol, a compound of formula (I) and etridiazole, a compound of formula (I) and famoxadone, a compound of formula (I) and fenamidone (RPA407213), a compound of formula (I) and fenarimol, a compound of formula (I) and fenbuconazole, a compound of formula (I) and fenfuram, a compound of formula (I) and fenhexamid (KBR2738), a compound of formula (I) and fenoxanil, a compound of formula (I) and fenpiclonil, a compound of formula (I) and fenpropidin, a compound of formula (I) and fenpropimorph, a compound of formula (I) and fentin acetate, a compound of formula (I) and fentin hydroxide, a compound of formula (I) and ferbam, a compound of formula (I) and ferimzone, a compound of formula (I) and fluazinam, a compound of formula (I) and fluopicolide, a compound of formula (I) and fludioxonil, a compound of formula (I) and fluoxastrobin, a compound of formula (I) and flumetover, a compound of formula (I) and SYP-LI90 (flumorph), a compound of formula (I) and fluopyram, a compound of formula (I) and fluoroimide, a compound of formula (I) and fluquinconazole, a compound of formula (I) and flusilazole, a compound of formula (I) and flusulfamide, a compound of formula (I) and flutolanil, a compound of formula (I) and flutriafol, a compound of formula (I) and folpet, a compound of formula (I) and fosetyl-aluminium, a compound of formula (I) and fuberidazole, a compound of formula (I) and furalaxyl, a compound of formula (I) and furametpyr, a compound of formula (I) and guazatine, a compound of formula (I) and hexaconazole, a compound of formula (I) and hydroxyisoxazole, a compound of formula (I) and hymexazole, a compound of formula (I) and IKF-916 (cyazofamid), a compound of formula (I) and imazalil, a compound of formula (I) and imibenconazole, a compound of formula (I) and iminoctadine, a compound of formula (I) and iminoctadine triacetate, a compound of formula (I) and ipconazole, a compound of formula (I) and iprobenfos, a compound of formula (I) and iprodione, a compound of formula (I) and iprovalicarb (SZX0722), a compound of formula (I) and isopropanyl butyl carbamate, a compound of formula (I) and isoprothiolane, a compound of formula (I) and kasugamycin, a compound of formula (I) and kresoxim-methyl, a compound of formula (I) and LY186054, a compound of formula (I) and LY211795, a compound of formula (I) and LY248908, a compound of formula (I) and maneb, a compound of formula (I) and mancopper, a compound of formula (I) and man-cozeb, a compound of formula (I) and mandipropamid, a compound of formula (I) and mefenoxam, a compound of formula (I) and mepanipyrim, a compound of formula (I) and mepronil, a compound of formula (I) and metalaxyl, a compound of formula (I) and metconazole, a compound of formula (I) and methasulfocarb, a compound of formula (I) and metiram, a compound of formula (I) and metiram-zinc, a compound of formula (I) and metominostrobin, a compound of formula (I) and metrafenone, a compound of formula (I) and myclobutanil, a compound of formula (I) and myclozoline, a compound of formula (I) and neoasozin, a compound of formula (I) and nickel dimethyldithiocarbamate, a compound of formula (I) and nitrothal-isopropyl, a compound of formula (I) and nuarimol, a compound of formula (I) and ofurace, a compound of formula (I) and organomercury compounds, a compound of formula (I) and orysastrobin, a compound of formula (I) and oxadixyl, a compound of formula (I) and oxasulfuron, a compound of formula (I) and oxine-copper, a compound of formula (I) and oxolinic acid, a compound of formula (I) and oxpoconazole, a compound of formula (I) and oxycarboxin, a compound of formula (I) and pefurazoate, a compound of formula (I) and penconazole, a compound of formula (I) and pencycuron, a compound of formula (I) and penthiopyrad, a compound of formula (I) and phenazin oxide, a compound of formula (I) and phosdiphen, a compound of formula (I) and phosphorus acids, a compound of formula (I) and phthalide, a compound of formula (I) and picoxystrobin (ZA1963), a compound of formula (I) and polyoxin D, a compound of formula (I) and polyram, a compound of formula (I) and probenazole, a compound of formula (I) and prochloraz, a compound of formula (I) and procymidone, a compound of formula (I) and propamocarb, a compound of formula (I) and propiconazole, a compound of formula (I) and propineb, a compound of formula (I) and propionic acid, a compound of formula (I) and proquinazid, a compound of formula (I) and prothioconazole, a compound of formula (I) and pyraclostrobin, a compound of formula (I) and pyrazophos, a compound of formula (I) and pyribencarb, a compound of formula (I) and pyrifenox, a compound of formula (I) and pyrimethanil, a compound of formula (I) and pyroquilon, a compound of formula (I) and pyr-oxyfur, a compound of formula (I) and pyrrolnitrin, a compound of formula (I) and quaternary ammonium compounds, a compound of formula (I) and quinomethionate, a compound of formula (I) and quinoxyfen, a compound of formula (I) and quintozene, a compound of formula (I) and silthiofam, a compound of formula (I) and simeconazole, a compound of formula (I) and sipconazole (F-155), a compound of formula (I) and sodium pentachlorophenate, a compound of formula (I) and spiroxamine, a compound of formula (I) and streptomycin, a compound of formula (I) and sulphur, a compound of formula (I) and tebuconazole, a compound of formula (I) and tecloftalam, a compound of formula (I) and tecnazene, a compound of formula (I) and tetraconazole, a compound of formula (I) and thiabendazole, a compound of formula (I) and thifluzamid, a compound of formula (I) and 2-(thiocyanomethylthio)benzothiazole, a compound of formula (I) and thiophanate-methyl, a compound of formula (I) and thiram, a compound of formula (I) and tiadinil, a compound of formula (I) and timibenconazole, a compound of formula (I) and tolclofos-methyl, a compound of formula (I) and tolylfluanid, a compound of formula (I) and triadimefon, a compound of formula (I) and triadimenol, a compound of formula (I) and triazbutil, a compound of formula (I) and triazoxide, a compound of formula (I) and tricyclazole, a compound of formula (I) and tridemorph, a compound of formula (I) and trifloxystrobin (CGA279202), a compound of formula (I) and triforine, a compound of formula (I) and triflumizole, a compound of formula (I) and triticonazole, a compound of formula (I) and validamycin A, a compound of formula (I) and vapam, a compound of formula (I) and valiphenal a compound of formula (I) and vinclozolin, a compound of formula (I) and zineb, a compound of formula (I) and ziram, a compound of formula (I) and zoxamide, a compound of formula (I) and 3-[5-(4-chlorophenyl)-2,3-dimethylisoxazolidin-3-yl]pyridine, a compound of formula (I) and 5-chloro-7-(4-methylpiperidine-1-yl)-6-(2,4,6-trifluorophenyl)[1,2,4]triazolo[1,5-a]pyrimidine and a compound of formula (I) and N-(4-chloro-2-nitrophenyl)-N-ethyl-4-methyl-benzsulfonamide.

Plant growth regulators (PGRs) affect growth and differentiation of plants.

More specifically, various plant growth regulators (PGRs) can, for example, reduce plant height, stimulate seed germination, induce flowering, darken leaf coloring, change the rate of plant growth and modify the timing and efficiency of fruiting.

In addition plant growth regulators (PGRs) may exhibit pronounced growth-regulating properties which can result in an increase in the yield of cultivated plants or harvested crops.

Further, plant growth regulators (PGRs) may have a growth inhibiting action which is dependent on the concentration. The growth of both monocots and dicots may be inhibited.

Inhibition of the vegetative growth of many cultivated plants permits more plants to be sown in a crop area, so that a higher yield may be obtained per unit of area. Inhibition of the vegetative growth of monocot plants, e.g. cultivated plants such as cereals, is sometimes desirable and advantageous. Such a growth inhibition is of economic interest.

The use of plant growth regulators (PGRs) for inhibiting the growth in height of cereals is also important, as shortening the stalks diminishes or completely eliminates the danger of lodging before harvesting.

In addition, plant growth regulators (PGRs) are able to bring about a strengthening of the stalks in crops of cereals and this too counteracts lodging.

Furthermore, the present invention also relates to compositions comprising the pyrrole derivatives of the present invention that improve plants, a process which is commonly and hereinafter referred to as “plant health”.

For example, advantageous properties that may be mentioned are improved crop characteristics including: emergence, crop yield, protein content, increased vigour, faster/delayed maturation, increased speed of seed emergence, improved nutrient utilization efficiency, improved nitrogen utilization efficiency, improved water use efficiency, improved oil content and/or quality, improved digestibility, faster/more even ripening, improved flavor, improved starch content, more developed root system (improved root growth), improved stress tolerance (e.g. against drought, heat, salt, light, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Advantageous properties, obtained especially from treated seeds, are e.g. improved germination and field establishment, better vigor, more homogeneous field establishment.

Advantageous properties, obtained especially from foliar and/or in-furrow application are e.g. improved plant growth and plant development, better growth, more tillers, greener leafes, largers leaves, more biomass, better roots, improved stress tolerance of the plants, more grain yield, more biomass harvested, improved quality of the harvest (content of fatty acids, metabolites, oil etc), more marketable products (e.g. improved size), improved process (e.g. longer shelf-life, better extraction of compounds), improved quality of seeds (for being seeded in the following seasons for seed production); or any other advantages familiar to a person skilled in the art.

It is therefore an object of the present invention to provide a method which solves the problems outlined above.

The present invention relates to plant-protecting active ingredients that are pyrrole compounds of formula (I) according to the invention, in particular the individual pyrrole compounds described in the above description as being preferred, and mixtures with increased efficacy and to a method of improving the health of plants by applying said compounds and mixtures to the plants or the locus thereof.

Compounds of the invention and for use in the methods of the invention can be made, for example, by following the reaction scheme and the methods detailed below. The starting materials used for the preparation of the compounds of the invention may be purchased from usual commercial suppliers or may be prepared by known methods. The starting materials as well as the intermediates may be purified before use in the next step by state of the art methodologies such as chromatography, crystallization, distillation and filtration.

N-substituted pyrroles can easily be prepared according to Acta Chemica Scandinavia, 1952, 6, 867

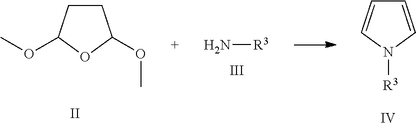

Bromination of IV with NBS will provide V according to Journal of Organic Chemistry, 1990, 55, 6317 or Tetrahedron, 1990, 31, 6785

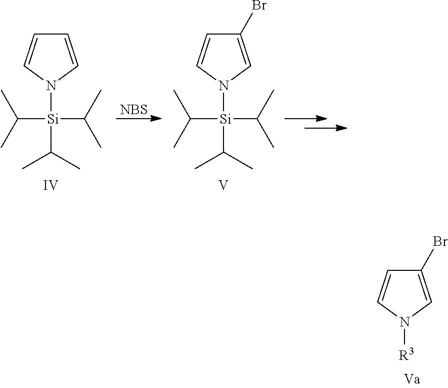

Metallation of the intermediate V in position 2 followed by trapping with an aldehyde will lead to compounds of type VI.

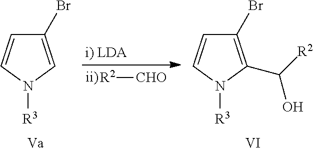

Conversion of VI to final compounds can be achieved following a procedure given in Synthesis, 2006, 22, 3883 or according to methods described in “Palladium in Heterocyclic Chemistry”, Jie Jack Li and Gordon W. Gribble; Pergamon 2000.

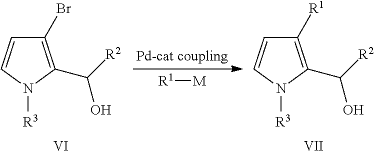

In Synthesis, 2006, 22, 3883 methods are described to prepare also 1-, 2-, 3- and 4-substituted pyrroles of generic formula (I):

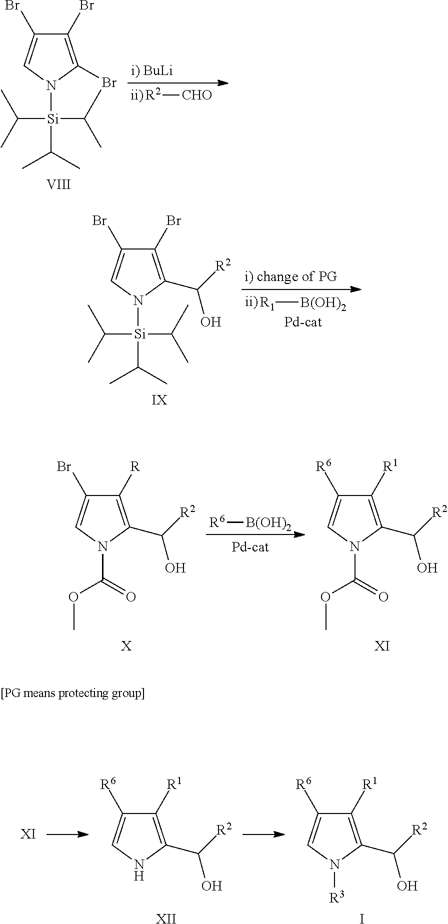

Alternative methods for the preparation of 1-, 2- and 3-substituted pyrroles can be found in Tetrahedron, 1996, 52, 6879

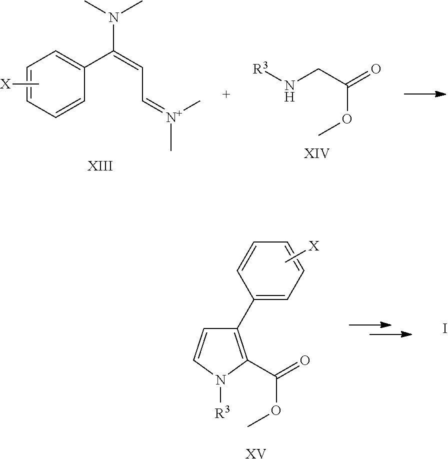

Examples of compounds for use in the present invention are shown below:

(I)

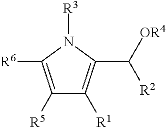

| No | R¹ | R² | R³ | R⁴ | R⁵ | R⁶ |
|---|---|---|---|---|---|---|
| 1 | 4-Cl—Ph | 3-Py | 4-Cl—Ph | H | H | H |
| 2 | 4-Cl—Ph | 3-Py | 4-Cl—Ph | H | H | Me |
| 3 | 4-Cl—Ph | 3-Py | 4-Cl—Ph | C(O)Me | H | H |
| 4 | 4-Cl—Ph | 3-Py | 4-Cl—Ph | H | Me | H |
| 5 | 4-Cl—Ph | 3-Py | 2-Cl—Ph | H | H | H |
| 6 | 4-Cl—Ph | 3-Py | 2-Cl—Ph | H | Et | H |
| 7 | 4-Cl—Ph | 3-Py | 3-Cl—Ph | H | H | H |
| 8 | 4-Cl—Ph | 3-Py | 3-Cl—Ph | H | 4-Cl—Ph | H |
| 9 | 2,4-Cl₂—Ph | 3-Py | 3-Cl—Ph | C(O)Et | H | H |
| 10 | 2,4-Cl₂—Ph | 3-Py | 4-Cl—Ph | H | H | H |
| 11 | 2,4-Cl₂—Ph | 3-Py | 4-Cl—Ph | H | Bu | H |
| 12 | 2,4-Cl₂—Ph | 3-Py | 2-Cl—Ph | H | H | H |
| 13 | 2,4-Cl₂—Ph | 3-Py | 3-Cl—Ph | H | H | H |
| 14 | 4-Cl—Ph | 3-Py | 2,4-Cl₂—Ph | H | H | H |
| 15 | 2-Cl—Ph | 3-Py | 2,4-Cl₂—Ph | H | H | H |
| 16 | 3-Cl—Ph | 3-Py | 2,4-Cl₂—Ph | H | H | H |
| 17 | 4-Cl—Ph | 5-Pyrimi | 4-Cl—Ph | H | H | H |
| 18 | 4-Cl—Ph | 5-Pyrimi | 2-Cl—Ph | H | H | H |
| 19 | 4-Cl—Ph | 5-Pyrimi | 3-Cl—Ph | H | H | H |
| 20 | 2,4-Cl₂—Ph | 5-Pyrimi | 4-Cl—Ph | H | H | H |
| 21 | 2,4-Cl₂—Ph | 5-Pyrimi | 2-Cl—Ph | H | H | H |
| 22 | 2,4-Cl₂—Ph | 5-Pyrimi | 3-Cl—Ph | H | H | H |
| 23 | 4-Cl—Ph | 5-Pyrimi | 2,4-Cl₂—Ph | H | H | H |
| 24 | 2-Cl—Ph | 5-Pyrimi | 2,4-Cl₂—Ph | H | H | H |
| 25 | 3-Cl—Ph | 5-Pyrimi | 2,4-Cl₂—Ph | H | H | H |
| 26 | 2-F,4-Cl—Ph | 5-Pyrimi | 4-Cl—Ph | H | H | H |
| 27 | 2-F,4-Cl—Ph | 5-Pyrimi | 2-Cl—Ph | H | H | H |
| 28 | 2-F,4-Cl—Ph | 5-Pyrimi | 3-Cl—Ph | H | H | H |
| 29 | 2-F,4-Cl—Ph | 3-Py | 4-Cl—Ph | H | H | H |
| 30 | 2-F,4-Cl—Ph | 3-Py | 2-Cl—Ph | H | H | H |
| 31 | 2-F,4-Cl—Ph | 3-Py | 3-Cl—Ph | H | H | H |
| 32 | 2,4-F₂—Ph | 5-Pyrimi | 4-Cl—Ph | H | H | H |
| 33 | 2,4-F₂—Ph | 5-Pyrimi | 2-Cl—Ph | H | H | H |
| 34 | 2,4-F₂—Ph | 5-Pyrimi | 3-Cl—Ph | H | H | H |
| 35 | 4-Cl—Ph | 4-F,3-Py | 2,4-Cl₂—Ph | H | H | H |
| 36 | 2-Cl—Ph | 4-Me,3-Py | 2,4-Cl₂—Ph | H | H | H |
| 37 | 3-Cl—Ph | 5-MeO,3-Py | 2,4-Cl₂—Ph | H | H | H |
| 38 | 4-Cl—Ph | 3-Py | 2,4-Cl₂—Ph | C(O)-c-Prop | H | H |
| 39 | 2-Cl—Ph | 3-Py | 2,4-Cl₂—Ph | H | H | Ph |
| 40 | 3-Cl—Ph | 3-Py | 2,4-Cl₂—Ph | H | Me | Me |
| 41 | 4-Cl—Ph | 4-Me,5-Pyrimi | 4-Cl—Ph | H | H | H |
| 42 | 4-Cl—Ph | 4-MeO,5-Pyrimi | 2-Cl—Ph | H | H | H |
| 43 | 4-Cl—Ph | 5-Pyrimi | 3-CF₃—Ph | H | H | H |
| 44 | 2-Thioph | 3-Py | 4-Cl—Ph | H | H | H |
| 45 | 4-Cl—Ph | 3-Py | 2-Thioph | H | H | H |
| 46 | 2-Fur | 3-Py | 2,4-Cl₂—Ph | H | H | H |
| 47 | 2,4-Cl₂—Ph | 3-Py | 2-Fur | H | H | H |
| 48 | 3-Fur | 3-Py | 2,4-Cl₂—Ph | H | H | H |
| 49 | 2,4-Cl₂—Ph | 3-Py | 3-Fur | H | H | H |
| 50 | c-Hx | 3-Py | 4-Cl—Ph | H | H | H |
| 51 | c-Hx | 3-Py | 2,4-F₂—Ph | H | H | H |
| 52 | 2-Cl—Ph | 3-Py | c-Hx | H | H | H |
| 53 | 2,4-Cl₂—Ph | 3-Py | c-Hx | H | H | H |
| 54 | i-Prop | 3-Py | 4-Cl—Ph | H | H | H |
| 55 | 2,4-Cl₂—Ph | 3-Py | i-Prop | H | H | H |
| 56 | 2,4-Cl₂—Ph | 3-Py | i-Amyl | H | H | H |
| 57 | 4-Cl—Ph | 3-Py | Et | H | H | H |
| 58 | 2,4-Cl₂—Ph | 3-Py | 4-Cl—Ph | H | CN | H |
| 59 | 2,4-Cl₂—Ph | 3-Py | 4-Cl—Ph | C(O)Me | CN | H |
| 60 | Bn | 3-Py | 4-Cl—Ph | H | CN | H |
| 61 | 2,4-Cl₂—Ph | 3-Py | 4-Cl—Ph | H | H | OMe |

In the above table, the following is meant by each abbreviation given for R¹ to R⁶:

| | |
|---|---|
| H | Hydrogen |
| CN | 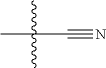 |
| Me | 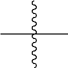 |
| Et | 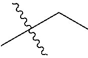 |
| Bu | 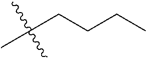 |
| i-Prop | 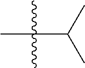 |
| i-Amyl | 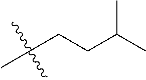 |
| OMe | 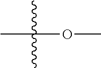 |
| C(O)Me | 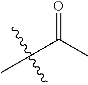 |
| C(O)Et | 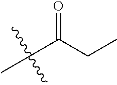 |
| C(O)-c-Prop | 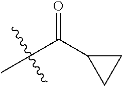 |
| c-Hx | 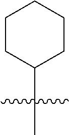 |
| Bn | 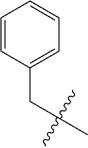 |
| Ph | 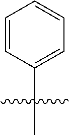 |
| 3-Py | 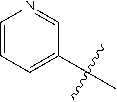 |
| 2-Fur | 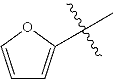 |
| 3-Fur | 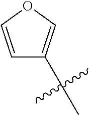 |
| 2-Thioph | 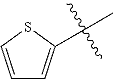 |
| 2-Cl—Ph | 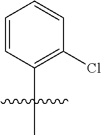 |
| 3-Cl—Ph | 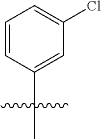 |
| 4-Cl—Ph | 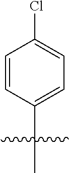 |
| 2,4-Cl₂—Ph | 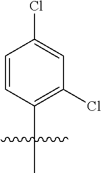 |
| 2-F,4-Cl—Ph | 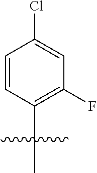 |
| 2,4-F₂—Ph | 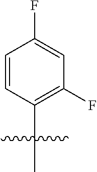 |
| 3-CF₃—Ph | 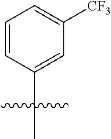 |
| 4-F,3-Py | 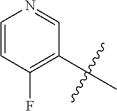 |
| 4-Me,3-Py | 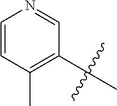 |
| 5-MeO,3-Py | 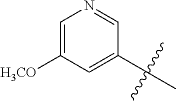 |
| 4-Me,5-Pyrimi | 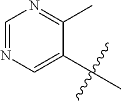 |
| 4-MeO,5-Pyrimi | 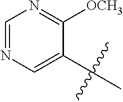 |
| 5-Pyrimi | 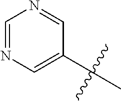 |

The pyrrole of the formula (I) can be obtained in different tautomeric forms.

Surprisingly, the pyrrole compounds of formula (I) according to the invention, in particular the individual pyrrole compounds above as being preferred, exhibit a plant growth regulator (PGR) activity. Therefore, the present invention also relates to the use of these pyrrole derivatives as plant growth regulators (PGRs).

The invention is illustrated by the following Examples:

Mass spectra data are obtained using LCMS: LC5: 254 nM—gradient 10% A to 100% B; A=H₂O+0.01% HCOOH; B=CH₃CN/CH₃OH+0.01% HCOOH positive electrospray 150-1000 m/z.

### EXAMPLE 1

### The preparation of [1,3-Bis-(3-chloro-phenyl)-1H-pyrrol-2-yl]-pyridin-3-yl-methanol

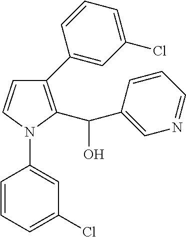

The title compound is prepared according to the following procedures:
Step A: 3-Chloroaniline (10.6 ml) and 2,5-dimethoxytetrahydrofuran (12.9 ml) are heated at 110° C. in acetic acid (100 ml) for 5 hours and 30 minutes. To the cooled reaction mixture, water (500 ml) is added and the reaction is stirred overnight. The brown precipitate formed is filtered and subsequently dissolved in dichloromethane, washed with aqueous sodium hydrogencarbonate (saturated) (100 ml), dried over sodium sulphate and concentrated in vacuo. 1-(3-Chloro-phenyl)-1H-pyrrole is isolated as black solid (35.2 g). ¹H-NMR (400 MHz, CDCl₃): 6.38 (t, 2H), 7.10 (t, 2H), 7.30 (m, 4H).
Step B: The compound obtained in Step A (24.9 g) is dissolved in N,N-dimethylformamide (280 ml). The reaction mixture is cooled at −67° C., and N-bromosuccinimide (24.9 g) is added portionwise. After 1 hour, the temperature is raised to −10° C. and the reaction stirred for further 60 minutes. The reaction mixture is partitioned between water (500 ml) and cyclohexane (300 ml) and after separation, the organic phase is washed with aqueous sodium hydrogencarbonate (saturated) (70 ml). The aqueous layer is extracted three times with cyclohexane (3×200 ml). The combined organic extracts are dried over sodium sulphate, concentrated in vacuo to obtain a brown oil (27.2 g) containing a mixture of 1-(3-Chloro-phenyl)-1H-pyrrole, 2-Bromo-1-(3-chloro-phenyl)-1H-pyrrole and 2,5-Dibromo-1-(3-chloro-phenyl)-1H-pyrrole in ratio 15:75:10, respectively. The mixture is used directly in the next step without further purification.
Step C: A solution of the crude mixture from Step B (27.2 g) and p-toluenesulfonic acid (2.0 g) in dichloromethane (200 ml) is stirred at room temperature for 3 hours. The reaction mixture is poured in an aqueous solution of sodium bicarbonate (saturated) (50 ml). Separation is performed and the organic phase is dried over sodium sulphate, concentrated in vacuo to afford 3-Bromo-1-(3-chloro-phenyl)-1H-pyrrole as red oil (25.6 g). ¹H-NMR (400 MHz, CDCl₃): 6.38 (m, 1H), 6.98 (m, 1H), 7.09 (m, 1H), 7.27 (m, 1H), 7.38 (m, 3H).
Step D: A solution of the compound obtained in Step C (10.3 g) in THF (20 ml) is slowly added to a solution of freshly prepared lithium 2,2,6,6-tetramethylpiperidide (1.2 equivalents) in tetrahydrofuran (80 ml), keeping the internal temperature below −70° C. The reaction mixture is stirred at −78° C. for 2 hours before adding N,N-dimethylformamide (9.3 ml). After further 2 hours at this temperature, the solution is warmed up to 0° C. and quenched by addition of an aqueous solution of ammonium chloride (saturated) (70 ml). Aqueous workup with ethyl acetate furnished a crude material which is purified by chromatography on silica gel (eluent: cyclohexane/ethylacetate=6:1). 3-Bromo-1-(3-chloro-phenyl)-1H-pyrrole-2-carbaldehyde is isolated as a pale yellow powder (4.7 g). MS (ES+) 286/288 (MH+); 1H-NMR (400 MHz, CDCl₃): 6.49 (d, 1H), 6.99 (m, 1H), 7.24 (m, 1H), 7.34 (m, 1H), 7.43 (m, 2H), 9.76 (s, 1H).
Step E: A suspension containing 0.29 g of the compound obtained from Step D, 3-chlorophenylboronic acid (0.39 g), barium hydroxide octahydrate (0.79 g) and [1,1′-bis(diphenylphosphino)-ferrocene]dichloropalladium (II) (1/1 complex with dichloromethane, 0.082 g) in a mixture of N,N-dimethylformamide and water (4/1, 5.0 ml, 0.2 M) is quickly warmed up to 110° C. with a preheated oil bath (120° C.). The reaction mixture is stirred at this temperature for 1 hour, and then cooled to room temperature. Water (20 ml) and ethyl acetate (20 ml) are added. The biphasic mixture is filtered over a short pad of Hyflo eluting with ethyl acetate. The filtrate is collected and the two layers separated. The organic phase is dried over sodium sulphate and evaporated in vacuo to afford a brown solid which is purified by column chromatography on silica gel (eluent: cyclohexane/ethylacetate=6:1). 1,3-Bis-(3-chloro-phenyl)-1H-pyrrole-2-carbaldehyde (0.30 g) is isolated as a yellowish powder. MS (ES+) 316/318 (MH+); ¹H-NMR (400 MHz, CDCl₃): 6.51 (m, 1H), 7.08 (m, 1H), 7.30 (m, 1H), 7.41 (m, 6H), 7.55 (m, 1H), 9.67 (s, 1H).
Step F:
Method A: n-Butyllithium (0.15 ml, 2.5 M in hexane) is added dropwise to a solution of 3-bromopyridine (0.04 ml) in diethyl ether (2.0 ml) at −78° C. After 15 min of stirring at this temperature, a solution of the compound obtained in Step E (0.10 g) in tetrahydrofuran (2 ml) is added. The reaction is stirred for 30 minutes and quenched by addition of an aqueous solution of ammonium chloride (saturated) (10 ml). The mixture is extracted with ethyl acetate (3×10 ml). The combined organic extracts are dried over sodium sulphate and concentrated in vacuo. The residue is purified by column chromatography on silica gel (eluent: cylohexane/ethylacetate=1:1) to afford the title compound, [1,3-Bis-(3-chloro-phenyl)-1H-pyrrol-2-yl]-pyridin-3-yl-methanol, as a pale yellow foam (0.090 g). MS (ES+) 395/397 (MH+); 1H-NMR (400 MHz, CDCl₃): 2.77 (bs, 1H), 6.03 (s, 1H), 6.31 (d, 1H), 6.69 (d, 1H), 6.89 (m, 1H), 7.00 (m, 1H), 7.14 (m, 7H), 7.38 (m, 1H), 8.05 (m, 1H), 8.17 (m, 1H).
Method B: Equimolecular amount (1.2 eq) of 3-bromopyridine and i-propylmagnesium chloride (2.0 M in tetrahydrofuran) are stirred in tetrahydrofuran (0.1 M) at 0° C. for 15 minutes and subsequently 10 minutes at room temperature. A tetrahydrofuran solution (0.1 M) of the compound isolated in Step E (1.0 eq) is added to the above slurry solution. The disappearance of the starting material is followed by TLC (eluent: cyclohexane/ethyl acetate=3:1). The crude reaction mixture is treated as described above.
Compounds of formula (X) (Table 2) and compounds of formula (XI) (Table 3) are prepared according to procedures analogous to those described in Example 1.

TABLE 2
Compounds of formula (X):
(X)

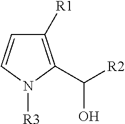

| | Corresponding | | | | | LC-MS | |
|---|---|---|---|---|---|---|---|
| No | N° in Table 1 | R¹ | R² | R³ | M.p. | (RT) | MS(ES+) |
| 62 | — | 3-Cl—Ph | 3-Py | 3-Cl—Ph | foam | 1.69 min | 395/397 |
| 63 | — | 2-Cl—Ph | 3-Py | 3-Cl—Ph | foam | 1.07 min | 395.1 |
| 64 | — | 4-CF₃—Ph | 3-Py | 3-Cl—Ph | foam | 1.30 min | 429.1 |
| 65 | 7 | 4-Cl—Ph | 3-Py | 3-Cl—Ph | foam | 1.18 min | 395.1 |
| 66 | — | 2-OMe—Ph | 3-Py | 3-Cl—Ph | foam | 0.96 min | 391.1 |
| 67 | — | 4-OMe—Ph | 3-Py | 3-Cl—Ph | foam | 1.02 min | 391.1 |
| 68 | — | 4-F—Ph | 3-Py | 3-Cl—Ph | foam | 1.06 min | 379.1 |
| 69 | — | 2-Me—Ph | 3-Py | 3-Cl—Ph | foam | 1.04 min | 375.1 |
| 70 | — | 4-Me—Ph | 3-Py | 3-Cl—Ph | foam | 1.11 min | 375.1 |
| 71 | — | 3-thioph | 3-Py | 3-Cl—Ph | foam | 0.92 min | 367.1 |
| 72 | — | 2-thioph | 3-Py | 3-Cl—Ph | foam | 0.96 min | 367.1 |
| 73 | — | 4-Br—Ph | 3-Py | 3-Cl—Ph | foam | 1.22 min | 439 |
| 74 | 13 | 2,4-Cl₂—Ph | 3-Py | 3-Cl—Ph | foam | 1.27 min | 429 |
| 75 | — | 2-F—Ph | 3-Py | 3-Cl—Ph | foam | 1.00 min | 379.1 |
| 76 | — | 2,4-F₂—Ph | 3-Py | 3-Cl—Ph | foam | 1.06 min | 397.1 |
| 77 | — | 3-CF₃—Ph | 3-Py | 3-Cl—Ph | foam | 1.29 min | 429.1 |
| 78 | — | 2-Cl-3-Py | 3-Py | 3-Cl—Ph | foam | 0.80 min | 396.1 |
| 79 | — | 5-Cl-2-thioph | 3-Py | 3-Cl—Ph | foam | 1.21 min | 401.1 |
| 80 | — | 2-Cl-4-OMe—Ph | 3-Py | 3-Cl—Ph | foam | 1.13 min | 425.1 |
| 81 | — | 2,4-Me₂—Ph | 3-Py | 3-Cl—Ph | foam | 1.16 min | 389.1 |
| 82 | — | 4-OCF₃—Ph | 3-Py | 3-Cl—Ph | foam | 1.33 min | 445.1 |
| 83 | — | 2-Me-4-Cl—Ph | 3-Py | 3-Cl—Ph | foam | 1.24 min | 409.1 |
| 84 | 31 | 2-F-4-Cl—Ph | 3-Py | 3-Cl—Ph | foam | 1.22 min | 413.0 |
| 85 | — | 2-Cl-4-F—Ph | 3-Py | 3-Cl—Ph | foam | 1.18 min | 413.0 |
| 87 | — | 2-OMe-3-Py | 3-Py | 3-Cl—Ph | foam | 1.42 min | 392.0 |

In the above table, the following is meant by each abbreviation given for R¹ to R³:

| | |
|---|---|
| 2-Cl—Ph | 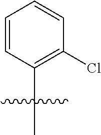 |
| 3-Cl—Ph | 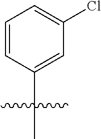 |
| 4-Cl—Ph | 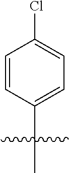 |
| 2-thioph | 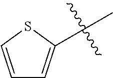 |
| 4-OMe—Ph | 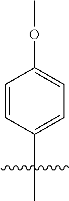 |
| 2,4-F₂—Ph | 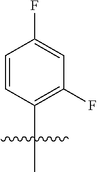 |
| 4-F—Ph | 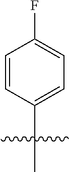 |
| 3-CF₃—Ph | 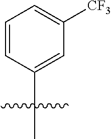 |
| 5-Cl-2-thioph | 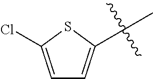 |
| 2-Cl-4-OMe—Ph | 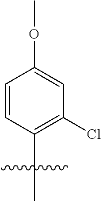 |
| 2-Me-4-Cl—Ph | 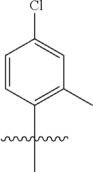 |
| 2-Cl-4-F—Ph | 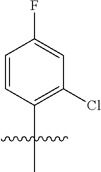 |
| 3-Py | 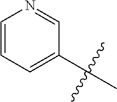 |
| 2-Me—Ph | 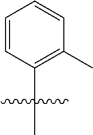 |
| 4-Me—Ph | 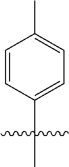 |
| 3-thioph | 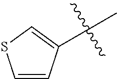 |
| 2-OMe—Ph | 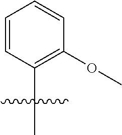 |
| 4-Br—Ph | 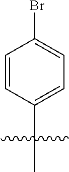 |
| 2,4-Cl₂—Ph | 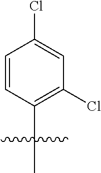 |
| 2-F—Ph | 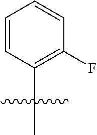 |
| 4-CF₃—Ph | 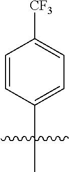 |
| 2-Cl-3-Py | 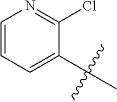 |
| 2,4-Me₂—Ph | 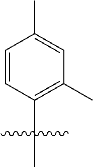 |
| 4-OCF₃—Ph | 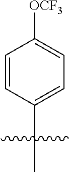 |
| 2-F-4-Cl—Ph | 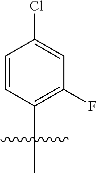 |
| 2-OMe-3-Py | 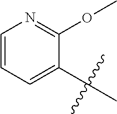 |

TABLE 3
Compounds of formula (XI):
(XI)

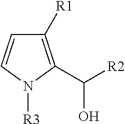

| | | | | | LC-MS | |
|---|---|---|---|---|---|---|
| No | R¹ | R² | R³ | M.p. | (RT) | MS(ES+) |
| 86 | n-Prop | 3-Py | 3-Cl—Ph | foam | 1.54 | 327/329 |
| | | | | | min | |

In the above table, the following is meant by each abbreviation given for R¹ to R³:

| | |
|---|---|
| n-Prop | 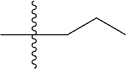 |
| 3-Cl—Ph | 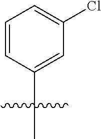 |
| 3-Py | 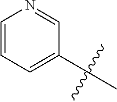 |

### Biological Evaluation

### EXAMPLE 2

### Biological Evaluation of Plant Growth Regulation Effects on Grape

5 weeks old grape seedlings cultivar (cv.) Gutedel were treated with the formulated test compound in a spray chamber. After an incubation period of 8 days at 22° C. and 80% relative humidity (r. h.) in a greenhouse, the plant growth regulating effects were assessed.
Compound 87 showed plant height decreased at 600 ppm.

### EXAMPLE 3

### Biological Evaluation of Plant Growth Regulation Effects on Wheat

2 weeks old wheat plants cultivar (cv.). Riband are treated with the formulated test compound in a spray chamber. After an incubation period of 8 days at 22° C. and 80% relative humidity (r. h.) in a greenhouse, the plant growth regulating effects are assessed.

1. Use of a compound of formula (I) 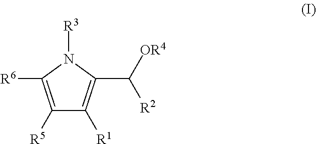
wherein
R¹ and R³ are, independently, hydrogen, or optionally substituted alkyl, alkenyl, alkynyl, heterocyclyl, trialkylsilyl, arylalkyl, aryloxyalkyl, arylthioalkyl, aryl or heteroaryl;
R² is optionally substituted alkyl, alkenyl, alkynyl, heterocyclyl, arylalkyl, aryl or heteroaryl;
R⁴ is H or acyl;
R⁵ and R⁶ are, independently, hydrogen, cyano, halogen or optionally substituted alkyl, alkenyl, alkynyl, heterocyclyl, alkoxy, alkoxycarbonyl, alkylthio, trialkylsilyl, arylalkyl, aryloxyalkyl, arylthioalkyl, aryl or heteroaryl;
or a salt or N-oxide thereof; as a plant growth regulator.

2. Use according to claim 1 wherein R¹ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

3. Use according to claim 2 wherein R¹ is selected from 2-chlorophenyl, 3-chlorophenyl, 4-chlorophenyl, 4-bromophenyl, 2-fluorophenyl, 4-fluorophenyl, 2,4-dichlorophenyl, 2,4-difluorophenyl, 2-fluoro-4-chlorophenyl, 2-chloro-4-fluorophenyl, 2-methylphenyl, 4-methylphenyl, 2,4-dimethylphenyl, 2-methoxyphenyl, 4-methoxyphenyl, 3-trifluoromethylphenyl, 4-trifluoromethylphenyl, 2-chloro-4-methoxyphenyl, 4-methoxytrifluomethylphenyl, 2-methyl-4-chlorophenyl, 2-chloro-3-pyridyl, 2-methoxy-3-pyridyl, 2-thienyl, 3-thienyl and 5-chloro-2-thienyl.

4. Use according to any one of claims 1 to 3 wherein R² is pyridyl or pyrimidinyl, each optionally substituted with halogen, C₁-C₆-alkyl, C₁-C₆-haloalkyl, C₁-C₆-alkoxy or C₁-C₆-haloalkoxy.

5. Use according to claim 4 wherein R² is 3-pyridyl.

6. Use according to any one of claims 1 to 5 wherein R³ is selected from hydrogen; C₁-C₆-alkyl optionally substituted with phenyl; phenyl optionally substituted with halogen, C₁-C₆-alkyl, C₁-C₆-haloalkyl, C₁-C₆-alkoxy or C₁-C₆-haloalkoxy; and a 5- or 6-membered heteroaryl optionally substituted with halogen, C₁-C₆-alkyl, C₁-C₆-haloalkyl, C₁-C₆-alkoxy or C₁-C₆-haloalkoxy.

7. Use according to claim 6 wherein R³ is selected from ethyl, isopropyl, isoamyl cyclohexyl; phenyl optionally substituted with 1 or 2 groups independently selected from chloro, fluoro and trifluoromethyl; and 2- or 3-furyl, and 2- or 3-thienyl, each optionally substituted with 1 or 2 chloro.

8. Use according to any one of claims 1 to 7 wherein R⁴ is H.

9. Use according to any one of claims 1 to 8 wherein R⁵ is selected from hydrogen, halogen, C₁-C₆-alkyl, C₁-C₆-alkoxy, cyano; and phenyl optionally substituted with halogen, C₁-C₆-alkyl, C₁-C₆-haloalkyl, C₁-C₆-alkoxy or C₁-C₆-haloalkoxy.

10. Use according to any one of claims 1 to 9 wherein R⁶ is selected from hydrogen, halogen, C₁-C₆-alkyl, C₁-C₆-alkoxy, cyano; and phenyl optionally substituted with halogen, C₁-C₆-alkyl, C₁-C₆-haloalkyl, C₁-C₆-alkoxy or C₁-C₆-haloalkoxy.

11. A compound, which is the (R)-enantiomer of the compound of formula (I) as defined in any one of claims 1 to 10; and salts thereof.

12. A compound, which is the (S)-enantiomer of the compound of formula (I) as defined in any one of claims 1 to 10; and salts thereof.

13. A method of regulating plant growth of crops of useful plants, which comprises applying to said plants, to one or more parts of said plants, or to the locus thereof or plant propagation material, a compound of formula (I) as defined in any one of claims 1 to 12.

14. A method according to claim 13, which comprises one or more applications of one of more compounds of formula (I) alone or in conjunction with one or more customary plant protection formulating auxiliaries.

15. A method according to claim 13 wherein two or more applications are carried out in sequence, and wherein the two or more applications have the same or different concentration or combinations of compounds of formula (I) or both.

16. A method according to any one of claims 13 to 15 wherein the useful crop plants are selected from the group consting of cereals, rice, beets, leguminous plants, oil plants, cucumber plants, fibre plants, vegetables, plantation crops, ornamentals, vines, bushberries, caneberries, cranberries, peppermint, rhubarb, spearmint, sugar cane and turf grasses.

17. A method according to any one of claims 13 to 16 wherein the plant growth regulating effect is an inhibition or a retardation of the plant growth.

18. An agricultural composition comprising a compound according to either claim 11 or claim 12, or an agrochemically acceptable salt thereof, and an agrochemically acceptable diluent or carrier.

19. An agricultural composition comprising one or more compounds of formula (I) as defined in any one of claims 1 to 12, and one or more customary plant protection auxiliaries.
